(19) United States
(12) Patent Application Publication
(10) Pub. No.: US 2021/0103936 A1
(43) Pub. Date: Apr. 8, 2021

(54) MEDICAL PRODUCT CONSISTENCY VERIFICATION

(71) Applicant: INTERNATIONAL BUSINESS MACHINES CORPORATION, ARMONK, NY (US)

(72) Inventors: EDSON GOMES PEREIRA, Cotia (BR); MARCELO MOTA MANHAES, Curitiba (BR); TIAGO DIAS GENEROSO, Poços de Caldas (BR); Marcel de Toledo Pineda, Santo Andre (BR); GLAUCIO ALEXANDRE DE AZEVEDO, Jardim Chapadão (BR); DANIEL DE PAULA TURCO, Campinas (BR)

(21) Appl. No.: 16/593,000

(22) Filed: Oct. 4, 2019

(51) Int. Cl. G06Q 30/00; G16H 20/10; G06F 17/27; G06F 17/18; G06N 3/08

(52) U.S. Cl. CPC G06Q 30/018; G16H 20/10; G06N 3/08; G06F 17/2785; G06F 17/18; G06F 17/271

(57) ABSTRACT

A method, computer system, and a computer program product for verifying the consistency of a current medical product is provided. The present invention may include generating a quantity associated with each of one or more active principles in the current medical product based on a plurality of current medical product data and a plurality of information from the data domains. The present invention may then include comparing the generated quantity associated with each of the one or more active principles in the current medical product with one or more constraints associated with a feasible solution associated with the current medical product. The present invention may further include determining a level of counterfeit risk based on the compared quantity associated with each of the one or more active principles in the current medical product with the one or more constraints from the feasible solution for the current medical product.

### BACKGROUND

The present invention relates generally to the field of computing, and more particularly to health care-related software and pharmacology.

Every region around the world, not only emerging countries, have been affected by the sale of falsified (i.e., substandard) medical products. Every year, the volume of falsified medical products increases worldwide. Falsified medical products may include the improper amount or quantity of one or more active pharmaceutical ingredients (APIs) and/or an inferior quality of one or more APIs or other components of the medical product. The use of such falsified medical products are illegal and may cause major health issues that may be harmful, and even fatal, to patients.

### SUMMARY

Embodiments of the present invention disclose a method, computer system, and a computer program product for verifying the consistency of a current medical product. The present invention may include generating a quantity associated with each of one or more active principles in the current medical product based on a plurality of current medical product data and a plurality of information from a plurality of data domains. The present invention may then include comparing the generated quantity associated with each of the one or more active principles in the current medical product with one or more constraints associated with a feasible solution associated with the current medical product. The present invention may further include determining a level of counterfeit risk based on the compared quantity associated with each of the one or more active principles in the current medical product with the one or more constraints from the feasible solution associated with the current medical product.

### BRIEF DESCRIPTION OF THE SEVERAL VIEWS OF THE DRAWINGS

These and other objects, features and advantages of the present invention will become apparent from the following detailed description of illustrative embodiments thereof, which is to be read in connection with the accompanying drawings. The various features of the drawings are not to scale as the illustrations are for clarity in facilitating one skilled in the art in understanding the invention in conjunction with the detailed description. In the drawings:

FIG. 1 illustrates a networked computer environment according to at least one embodiment;

FIG. 2 is an operational flowchart illustrating a process for verifying the consistency of a medical product according to at least one embodiment;

FIG. 3 is an operational flowchart illustrating a process for engineering data according to at least one embodiment;

FIG. 4 is an exemplary graphical representation illustrating for a feasible solution for multiple active principles in a medical product from an optimization engine during the optimization phase in according to at least one embodiment;

FIG. 5 is a block diagram of internal and external components of computers and servers depicted in FIG. 1 according to at least one embodiment;

FIG. 6 is a block diagram of an illustrative cloud computing environment including the computer system depicted in FIG. 1, in accordance with an embodiment of the present disclosure; and

FIG. 7 is a block diagram of functional layers of the illustrative cloud computing environment of FIG. 6, in accordance with an embodiment of the present disclosure.

### DETAILED DESCRIPTION

Detailed embodiments of the claimed structures and methods are disclosed herein; however, it can be understood that the disclosed embodiments are merely illustrative of the claimed structures and methods that may be embodied in various forms. This invention may, however, be embodied in many different forms and should not be construed as limited to the exemplary embodiments set forth herein. Rather, these exemplary embodiments are provided so that this disclosure will be thorough and complete and will fully convey the scope of this invention to those skilled in the art. In the description, details of well-known features and techniques may be omitted to avoid unnecessarily obscuring the presented embodiments.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language, Python programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The following described exemplary embodiments provide a system, method and program product for verifying the consistency of a medical product. As such, the present embodiment has the capacity to improve the technical fields of health care-related software and pharmacology by verifying the consistency of a medical product (i.e., drug, medication) based on the quantity and/or quality of the active principle. More specifically, the consistency verification program may identify a particular medical product. Then, data associated with the medical product may be collected and analyzed to determine the quantity and/or quality of the active principle(s) included in the medical product. Such quantity and/or quality of the active principle(s) in the medical product may then be compared with a feasible solution of the medical product to determine whether the medical product has a high or low risk of counterfeit. Depending on the counterfeit risk and any discrepancies, the consistency verification program may then notify the appropriate authorities of such findings.

As previously described, every region around the world, not only emerging countries, have been affected by the sale of falsified/substandard medical products. Every year, the volume of falsified medical products increases worldwide. Falsified medical products may include the improper amount or quantity of one or more active pharmaceutical ingredients (APIs) and/or an inferior quality of one or more APIs or other components of the medical product. The use of such falsified medical products are illegal and may cause major health issues that may be harmful, and even fatal, to patients. Approximately, there have been 72,000 to 169,000 pneumonia-related children deaths per year that may be attributed to falsified antibiotics according to the University of Edinburgh. In addition, more than 250,000 children death per year may be linked to the increase amount of falsified (i.e., counterfeit) medical products, ranging from inferior quality vaccines to falsified medications with an improper amount (or none) of the active principle in the original medical product, that are used by children to prevent acute infections and/or diseases, such as hepatitis, yellow fever and meningitis.

Approximately, one in 10 medical products sold in low/middle income countries may be falsified, with expensive and high-demand medical products as a common target for counterfeiters. Approximately, half of the prescriptions in the United States may be filled with approved generic medical products. However, these approved generic medical products may be falsified and may be confused with counterfeit medical products. Additionally, the inferior quality of medical products may increase antibiotic resistance, which poses even more risks to patients. As such, the World Health Organization (WHO) estimates the global counterfeiting medical product trade may be worth over $250 billion United States dollars (USD) thereby leading health professionals to now consider falsified medical products as potential reason for a patient to have an unexpected response and/or side effect to a medical product.

However, falsified medical products may be difficult to detect since the counterfeit medical products are designed to appear identical to the original medical product. As such, the problem may be to ensure that the active pharmaceutical ingredient (API) used in the manufacturing is coming from a trusted provider capable to extract and manipulate the proper components in the proper quantity and/or quality. Therefore, it may be advantageous to, among other things, verify the amount (if any) of raw material (i.e., active principle) or key components in a medical product and comparing that amount with the amount of medical product produced to determine whether the medical product may be effective (i.e., feasible, likelihood of producing the intended results), as well as evaluating the details associated with the key components, verifying the industry capacity to produce those key components at the appropriate quality and quantity to thereby validate the consistence of production flow and determine if there is a counterfeit risk.

According to at least one embodiment, the consistency verification program may verify if the amount of raw material (active principle), when compared with the amount of medical product produced, is effective. The consistency verification program may further verify the key components utilized to produce the medical products, evaluate the key component details, check the industry capacity to produce those key components to confirm and assure the industry is capable to produce the volume according to the components delivered. The consistency verification program may further analyze information (i.e., dimensions) collected by the Internet of Things (IoT), websites and other sources, and extract the meaning of the information collected by utilizing feature extraction techniques. Then, the consistency verification program may utilize natural language processing (NLP) and visual recognition for non-structured data for data processing. Then, utilizing the supervised machine learning (ML) model separated for each dimension and the optimization engine, the consistency verification program may identify if the counterfeit risk (e.g., risk of fraud) is high or low. In the present embodiment, each supervised ML model may be trained separately to improve results, and the optimization engine may be used to reduce failures and improve the precision of the consistency verification program.

According to at least one embodiment, the consistency verification program may produce data associated with the Internet of Things (IoT), weather, property size, volume of raw material and other key components associated with a medical product from various sources (e.g., fiscal validation, taxes, tributes, contract agreement, fiscal data about producers, financial transaction, and goods productivity) in several dimensions against scientific articles and government regulations. With such data, the consistency verification program may validate the consistency of the production flow and may determine the level of risk for such a medical product to be a counterfeit.

According to at least one embodiment, the consistency verification program may include a data engineering phase, which includes a data wrangling stage (i.e., data extraction stage), a data cleansing stage, and a data preparation (i.e., data normalization) stage. During the data wrangling stage, the consistency verification program may extract the unstructured data by executing a crawl component that extracts data from different information source producers (e.g., IoT, websites, images of medicine leaflets). The consistency verification program may then extract corresponding context of the extracted data by executing one or more feature extraction techniques to collect the context of the data by using NLP techniques and visual recognition techniques of a machine learning (ML) model. Such ML models may be trained based on subject matter experts (SMEs) that extract the context of unstructured data. The extracted data and corresponding context may be merged into a data set. During the data cleansing stage, the consistency verification program may cleanse the data to eliminate any inconsistencies in the record and any invalid data. The resulting cleansed data may be syntactically correct and semantically correct without outliers. During the data preparation stage, the data may be transformed or consolidated on each dimension for a valid input to a supervised ML model in which each dimension may include input data and the ML model to be trained.

According to at least one embodiment, the consistency verification program may then include a training phase in which artificial intelligence (AI) or ML models may be trained. The ML models may be trained from the data engineering phase on each dimension and the algorithm may be a logistic regression or a neural network depending on the number of features on each dimension. The output of each model dimension may include coefficients in a regression model. For example, the coefficient for a goods productivity would output the amount of active principle limits acceptable considering the scientific articles and regulatory agencies. The output of values on each model dimension may be input for the next phase, an optimization phase.

According to at least one embodiment, the consistency verification program may include an optimization phase. During the optimization phase, the values of each dimension of ML models may be included as input into linear functions. As such, the consistency verification program may correlate the active principles into some constraints. The output may be the objective function and constraints. A new number of active principle may be received by the consistency verification program from the user input that may be compared against linear programming function to analyze if the active principle amount related is inside the boundaries. If the new number of active principle is inside a feasible (i.e., effective) solution area, then there may be a low risk of counterfeiting medical products. Otherwise, if the active principle amount related is outside the feasible solution, then there may be a high risk. The determined risk of counterfeiting may then be displayed in a dashboard for the user and may be a source of information for further investigation.

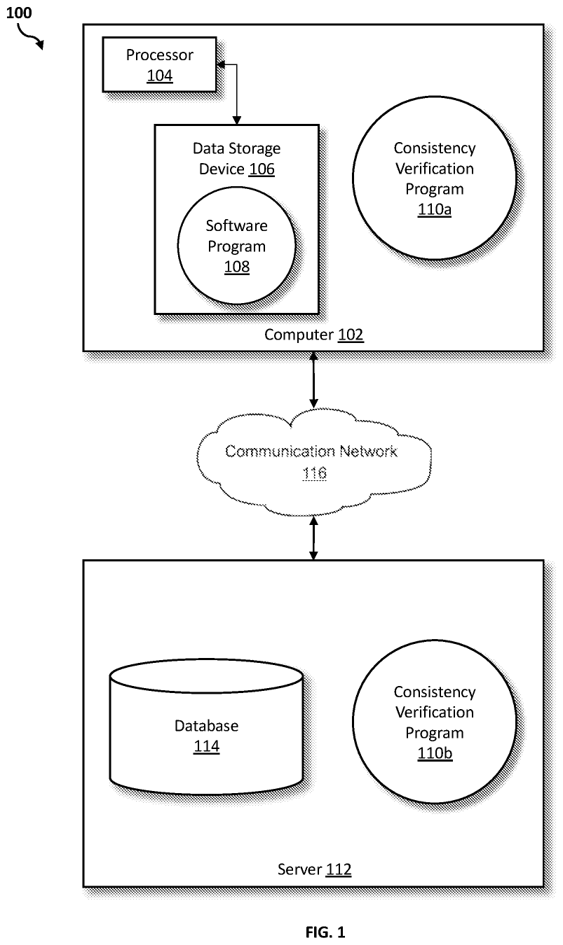

Referring to FIG. 1, an exemplary networked computer environment 100 in accordance with one embodiment is depicted. The networked computer environment 100 may include a computer 102 with a processor 104 and a data storage device 106 that is enabled to run a software program 108 and a consistency verification program 110a. The networked computer environment 100 may also include a server 112 that is enabled to run a consistency verification program 110b that may interact with a database 114 and a communication network 116. The networked computer environment 100 may include a plurality of computers 102 and servers 112, only one of which is shown. The communication network 116 may include various types of communication networks, such as a wide area network (WAN), local area network (LAN), a telecommunication network, a wireless network, a public switched network and/or a satellite network. It should be appreciated that FIG. 1 provides only an illustration of one implementation and does not imply any limitations with regard to the environments in which different embodiments may be implemented. Many modifications to the depicted environments may be made based on design and implementation requirements.

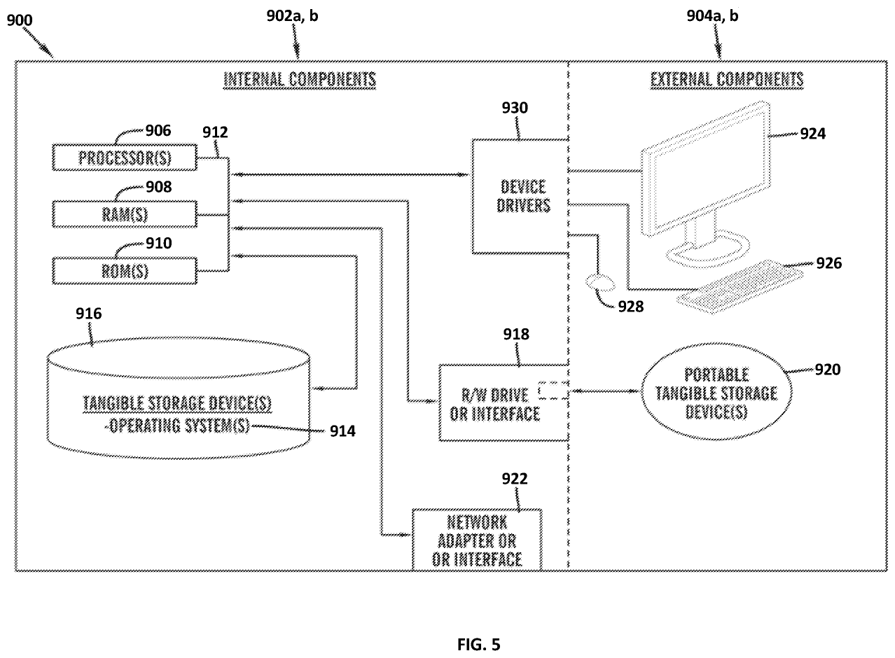

The client computer 102 may communicate with the server computer 112 via the communications network 116. The communications network 116 may include connections, such as wire, wireless communication links, or fiber optic cables. As will be discussed with reference to FIG. 5, server computer 112 may include internal components 902a and external components 904a, respectively, and client computer 102 may include internal components 902b and external components 904b, respectively. Server computer 112 may also operate in a cloud computing service model, such as Software as a Service (SaaS), Analytics as a Service (AaaS), Platform as a Service (PaaS), or Infrastructure as a Service (IaaS). Server 112 may also be located in a cloud computing deployment model, such as a private cloud, community cloud, public cloud, or hybrid cloud. Client computer 102 may be, for example, a mobile device, a telephone, a personal digital assistant, a netbook, a laptop computer, a tablet computer, a desktop computer, or any type of computing devices capable of running a program, accessing a network, and accessing a database 114. According to various implementations of the present embodiment, the consistency verification program 110a, 110b may interact with a database 114 that may be embedded in various storage devices, such as, but not limited to a computer/mobile device 102, a networked server 112, or a cloud storage service.

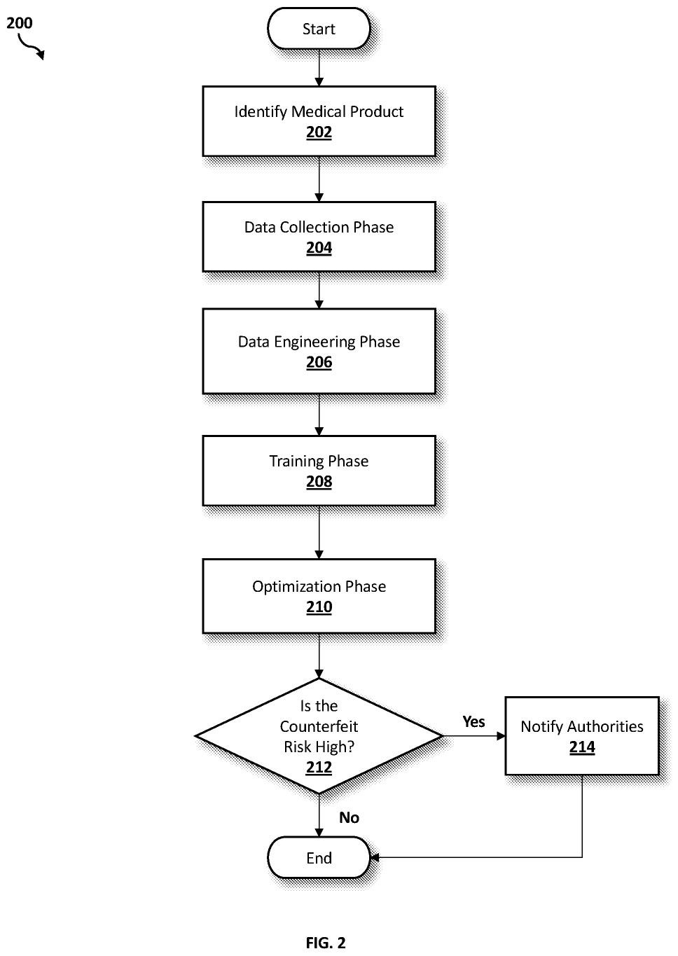

According to the present embodiment, a user using a client computer 102 or a server computer 112 may use the consistency verification program 110a, 110b (respectively) to verify the consistency of a medical product based on the quantity and/or quality of the active principle. The consistency verification method is explained in more detail below with respect to FIGS. 2, 3 and 4.

Referring now to FIG. 2, an operational flowchart illustrating the exemplary medical product consistency verification process 200 used by the consistency verification program 110a, 110b according to at least one embodiment is depicted.

At 202, a medical product is identified. Utilizing a software program 108 on the user's device (e.g., user's computer 102), data associated with a medical product (i.e., current medical product data) may be transmitted as input into the consistency verification program 110a, 110b via the communication network 116. The data may include the name of the medical product (e.g., generic, brand), administered location (e.g., location in which the medical product was administered to the patient, namely pharmacy, medical facility, medical provider), date of administration (e.g., date in which the prescribed was filled, or vaccination administered to the patient), and any other identifiers associated with the medical product.

In at least one embodiment, each container or packaging associated with the medical product may include a bar code, which the user may scan, via a camera or other scanning device associated with the user device, to upload the current medical product data. In some other embodiments, the container or packaging associated with the medical product may include an identification number, which the user may enter into the consistency verification program 110a, 110b, to retrieve the current medical product data.

For example, Patient Z has a family history of genetic cancer for past several generations. Several members of Patient Z's family has suffered from stomach cancer. Unfortunately, Patient Z started to experience symptoms of stomach cancer, namely severe and persistent stomach pain, nausea and severe bloating after eating. After consulting a physician and undergoing a biopsy, Patient Z was diagnosed with stomach cancer, and immediately started treatment, included Medication XYZ to fight the cancerous cells. After a week of taking Medication XYZ twice a day, her stomach pain worsened which was not a part of the desired results. As such, Patient Z's oncologist insisted that she immediately stop taking Medication XYZ. The oncologist then submits the current medical product data associated with Medication XYZ to a lab. At the lab, the User Z transmits the current medical product data into the consistency verification program 110a, 110b by scanning the barcode on the side of packaging for Medication XYZ.

Next, at 204, the data collection phase is commenced. During the data collection phase, data may be collected from different sources, and stored in different data domains (e.g., database 114), to identify the capacity of production for the components utilized during the manufacturing process. The data may be transmitted as input into the consistency verification program 110a, 110b via the communications network 116 by utilizing a software program 108 on the user's device (e.g., user's computer 102).

The data, stored in the data domains, may include, namely pharmaceutical data (e.g., public information about the components used to produce a medical product), agreement data (e.g., public data about international and national agreements and/or treaties involving countries and limitations for International Drug Control and strike/block), producer data (e.g., companies or producers responsible for collecting and/or manage the basic components from natural sources or manipulates in laboratories), scientific data (e.g., from scientific articles and/or publications that involves new discovery announcements about new components, drugs impacting the usage or consummation of pharmaceutical goods to improve public health and treatments and/or to produce benefits. Comments and/or trusted blog posts from communities looking for specific products and/or different methods to produce the medical product or extract the raw materials associated with the medical product), news data (e.g., announcements associated with the quantity and/or quality of the medical product sold during a specific period that may be compared with the quality and/or quantity produced to indicate whether there may be a discrepancy), public sector data (e.g., announcements for the local government and/or organization responsible for auditing and controlling the producers and logistics for extraction or production of ingredients and components to be used in the industry), and the Food and Drug Agencies data (e.g., local organization that may define the patterns and registry for the medical products and the components used to create the medical products). In at least one embodiment, the producer data may include regularly published financial reports and each transaction that may be implied in taxes and financial transactions to describe the productivity and expansion capacity.

In at least one embodiment, the consistency verification program 110a, 110b may utilize a search engine to parse through different trusted websites for data associated with the medical product. The search engine may utilize natural language processing (NLP) techniques (e.g., key words) to identify an article, publication and/or information (i.e., information) associated with the medical product on a trusted website. The identified information may then be stored in an appropriate data domain based on the source of the information, and/or the context of the information. For example, if the information includes the empirical formula for Medical Product X, then that scientific data will be stored in the goods productivity data domain.

In some embodiments, the user may be alerted of any suspicious or conflicting information retrieved by the search engine and stored in one or more data domains. In at least one embodiment, such suspicious or conflicting information may be indicated with a flag or label in the data domain. As such, when such information is transmitted, the flag or label indicated that such information is suspicious, or conflicting may be included. In at least one other embodiment, the recently retrieved suspicious or conflicting information may be placed in another database (e.g., database 114) with the corresponding previously stored information (e.g., information that such information conflicts with). As such, the user may be prompted (e.g., via a dialog box) that such conflicting information exists and may be excluded when verifying the consistency of the medical product based on the quantity and/or quality of the active principle. In one embodiment, the user may manually search and determine the reason for the conflicting information, and may then eliminate one or both conflicting information on the medical product. In one other embodiment, the consistency verification program 110a, 110b may automatically utilize the search engine to parse additional trusted websites to resolve the conflicting information by determining whether the source of either the recently retrieved or previously retrieved information is invalid, based on the comments associated with the article and/or publication on the information, or any other valid method of resolving the conflict.

In the at least one embodiment, the starting point of the consistency verification program 110a, 110b may be data that is available to the general public, such as basic components and quantities used to manipulate the medical product.

The data may be transmitted to various data domains, including the source productivity domain, fiscal validation domain, tax and tributes domain, contract agreement domain, fiscal validation domain, financial transaction domain, goods productivity domain, based on the medical product identified.

The source productivity data domain may utilize a large diversity of data from Internet of Things (IoT), weather, property size and historical data associated with the production of a particular medical product. In addition, the source productivity data domain may include the volume (e.g., quantity) of raw material utilized to produce a particular medical product. Based on the source productivity data domain, the user may retrieve producer data that may be utilized to determine the volume of the raw materials, including active pharmaceutical ingredients (APIs), for the original medical product to be effective.

The fiscal validation data domain may include any documents utilized to send and/or transport the medical products, as well as any documents utilized to notify one or more government agencies about the commercial transaction associated with a particular medical product. As such, the user may verify the substance and terms of the commercial transaction associated with the medical product. The tax and tributes data domain may include any commercial transactions that are subject to tax payment for the government. Therefore, based on the tax and tributes data domain, the user may verify with local laws that the taxes paid associated with the medical product, which may be utilized to measure the volume of the APIs in the medical product.

The contract agreement data domain may involve several parts including the producers, distributors and other key partners in the stream of commerce associated with the medical product, and the APIs associated with the medical product. The contract agreement data domain may include the terms (e.g., purchase price, quantity of the raw material, name and source of the raw material) associated with the contracts and/or agreements with any key partners in the stream of commerce associated with the medical product and/or the APIs associated with the medical product. The information generated from the contract agreement data domain may be checked for any discrepancies in the medical product (e.g., issues with the quality and/or quality of the raw materials) to determine if there is a risk of counterfeiting for the medical product. For example, if the contract between Producer A and Distributor A is for glucosamine instead of paracetamol, an API for acetaminophen, the contract agreement domain may verify that there is a discrepancy in the type of API, namely paracetamol, in the batch of the acetaminophen produced by Producer A.

The fiscal validation data domain may include any documents indicating the quantity of raw materials and/or medical product received by key partners in the stream of commerce, and the quantity of raw materials in storage. As such, the user may verify that the quantity of raw materials and/or medical products received by a key partner matches the quantity of raw materials included in storage. In at least one embodiment, the fiscal validation data domain may include a reason for any discrepancy in the retrieval and storage of the medical product and/or raw materials associated with the medical product. Such a reason may be included in the documents and disclosed to the user. In some embodiments, the details associated with the discrepancy may be confidential and/or classified. In such an instance, the fiscal validation data domain may indicate that there is a reason for the discrepancy, and further note that details of such reason are confidential and/or classified as well as including the government agency or entity that determined and/or implemented such a confidential and/or classified label.

The financial transaction data domain may include any documents (e.g., invoices, bills of lading, shipping slips) confirming that the commercial transaction agreed in the contract and/or agreement (e.g., from the contract agreement domain) were paid, either full payment or partial payment received. The financial transaction data domain may analyze the financial accounts of the key partners in the stream of commerce for the medical product to confirm the payment in accordance with the contract between the key partners.

The goods productivity data domain may include the formula (e.g., chemical, empirical and molecular) published on the medical product based on scientific research and publications. As such, the user may compare the formula from the goods productivity data domain with the formula of the medical product received to determine whether the quantities and APIs match.

In another embodiment, the user may add and/or delete data domains based on any additional information retrieved on the raw materials, medical product and/or the pharmacology industry. Subject matter experts (SMEs) may be utilized to review and analyze such additional information retrieved on the raw materials, medical product and/or pharmacology industry to determine whether data domains may be added and/or deleted.

The consistency verification program 110a, 110b may manage and maintain each of the data domains, by consolidating data on each dimension of the data domains on a regular basis, under the supervision and direction of SMEs. Machine learning (ML) techniques may be utilized to train and validate each data domain in which the SMEs may select a feature (i.e., a dimension) and may validate the performance of each data domains. The SMEs may further test each data domains through the use of multiple validation model in which scores may be assigned to each data domain based on the precision of each data domain thereby normalizing accuracy. Based on the findings of the SMEs, each data domain and corresponding artificial intelligence (AI) model for each data domain may be retrained in the training phase as described below.

In at least one embodiment, the consistency verification program 110a, 110b may include data domains that transmit data from global sources (e.g., sources located in every region around the world). In some embodiments, the user may configure the settings for the data domains to transmit data from a specific location or source (e.g., limit data retrieval to the raw materials and/or medical products for sale in the United States, or a specific United States state or region).

Continuing with the previous example, the consistency verification program 110a, 110b identifies data derived from different sources and stored on the data domains, namely core components used to assemble pharmaceutical products across the supply chain and tracks the entire stream of commerce for Medication XYZ until Medication XYZ is assembled and/or manufactured.

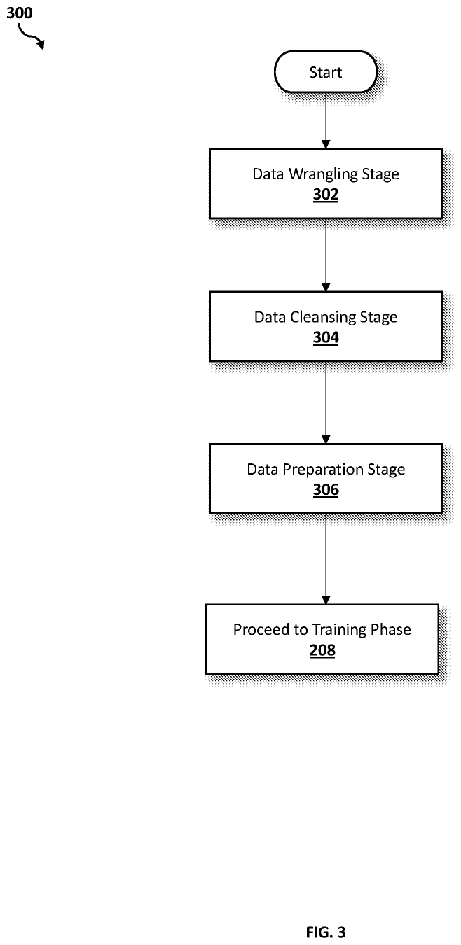

Then, at 206, the data engineering phase is commenced. The data engineering phase may include three different stages: (1) a data wrangling stage; (2) a data cleansing stage; and (3) a data preparation stage. During the data wrangling stage, the data associated with the current medical product data may be extracted, by a crawl component, from the data domains. Then, during the data cleansing stage, the extracted data may be cleansed to eliminate inconsistent and invalid data. Then, during the data preparation stage, the cleansing data may be normalized for input in a ML model supervised. The exemplary data engineering process 300 for the collected data, including the data wrangling stage, data cleansing stage, and data preparation stage, will be described in greater detail below with respect to FIG. 3.

Continuing with the previous example, the consistency verification program 110a, 110b identifies and extracts data and information associated with the active principles of Medication XYZ, Components Z and Y. The consistency verification program 110a, 110b identifies that Company YZ bought 1000 kg of each active principle, Component Z and Component Y, which may be used to produce 10 million pills (taking into account that there is an acceptable loss in the production process). However, Company YZ sold 15 million pills to the market. As such, there may be less quantity of the active principles or no active principles in the assembled and/or manufactured Medication XYZ.

In another embodiment, if a discrepancy is noticed by the consistency verification program 110a, 110b from the data obtained from different sources and stored in the data domains, then the consistency verification program 110a, 110b may alert the user of such discrepancies. The user may then elect to notify the authorities immediately for further investigation. For example, if the consistency verification program 110a, 110b notices that 200,000 vaccines for measles, mumps and rubella (MMR) fail to include multiple active principles, then the user may decide to immediately notify multiple government agencies as the consistency verification program 110a, 110b continues to run.

Then, at 208, the training phase is commenced. To train a ML model, training data can be fed as input into a learning algorithm. The training data may include features (i.e., individual independent variables that act as the input), and each feature is a dimension. The greater the number of features (or dimensions), the higher the level of complexity of the trained ML model, which may lead to a slower computation by the trained ML model. The learning algorithm may analyze the training data to find patterns (i.e., hidden or unhidden) in the training data, such that the input parameters correspond to the target (i.e., output of the input variables). The output of training the ML model may include a ML model that may be used to make predictions. As such, the normalized (or consolidated) data associated with each dimension for each data domain (i.e., results from the data engineering phase) may be input as training data into a logistic regression. The output values of the logistic regression may be coefficients per dimension, for example:

$$\frac{1}{1 + {e}^{- {∅}^{{γ}_{x}}}}$$

Where Ø^{γ}x represents the values for active principles associated with the medical product.

As the output of the logistic regression, the coefficient per dimension for each data domain may be utilized to train the final ML model that may provide numeric values associated with the quantity of active principle for the medical product. For example, the output is the acceptable quantity limits of active principle considering the scientific articles, regulatory agencies, or other sources associated with the medical product.

In at least one embodiment, linear regression may be utilized as follows:
Consider X₁,X₂,X₃, . . . Xₙ are the active principles.
X₁,X₂,X₃, . . . Xₙ are continuous and X₁,X₂,X₃, . . . Xₙ>=0

The linear regression model may utilize the following linear equations:

d₁₁*x₁+d₁₂*x₂+d₁₃*x₃+d₁₄*x₄+ . . . d₁ₙ*xₙ<=e₁

d₂₁*x₂+d₂₂*x₂+d₂₃*x₃+d₂₄*x₄+ . . . d₂ₙ*xₙ<=e₂

where the coefficients matrix d(mn) are collected from AI models and describes the combinations of active principles on the referred product, and e₁ and e₂ represent the maximum amount viable of those combinations.

Continuing with the previous example, the data from each data domains is fed into separate AI models and determines that the two active principles in Medication XYZ were calculated as Component Y at 150 mg and Component Z at 140 mg.

Then, at 210, the optimization phase is commenced. During the optimization phase of the consistency verification program 110a, 110b, the values of each dimension of the ML model may be utilized to determine whether the active principle of the current medical product is within standard boundaries for that medical product (i.e., feasible solution). First, the consistency verification program 110a, 110b may transmit as input the coefficients provided from each dimension of the ML model in the training phase into an objective function and constraints module via the communications network 116. The coefficients may be utilized to build the objective function considering the medical product may be expressed by variable x, which represents the source of the medical product from 1 to n as >x₁ . . . , xₙ.

To minimize/maximize production of a medical product in an objective function:

$${c}_{0} + {c}_{1} {x}_{1} + {c}_{2} {x}_{2} + {c}_{3} {x}_{3} + … + {c}_{n} {x}_{n} = {c}^{T} x$$

$${a}_{1} <= {x}_{1} <= {b}_{1}$$

$${a}_{2} <= {x}_{2} <= {b}_{2}$$

$${a}_{3} <= {x}_{3} < = {b}_{3}$$

$$…$$

$${a}_{n} < = {x}_{n} < = {b}_{n}$$

where a₁,a₂,a₃ . . . an are the lower bound values delivery by AI models and b₁,b₂,b₃ . . . bₙ are the upper bound values delivery by AI models in a linear regression model.

The constraints of the source of the medical product may be:

x₁<=b₁

x₂<=b₂

xₙ<=bₙ

where b₁, b₂, . . . bₙ may the feasible amount of source of medical products delivered by the trained ML model.

For example, x₁<=300 mg represents less than or equal to 300 mg for Medical Product A. Therefore, x₁ cannot be more than 300 mg. The value, 300 mg, was calculated by a combination of coefficients delivered by multiple dimensions associated with multiple data domains from the previously trained ML models. This constraint may for x₁ may be utilized by the objective function and constraints module.

Then, the objective function and constraints module linear may utilize an optimization engine to minimize the risk of fraud (i.e., counterfeit risk) based on whether the quantity of active principle falls within the range of standard medical product (i.e., feasible solution). The optimization engine may utilize linear functions, where, for example, c^{T}x where the variable “c” represents the coefficients matrix generated by AI models of each active principle multiplied per transpose matrix of variables that are the active principles to correlate the active principles into one or more constraints. As such, the quantity of active principle from the current medical product data may be compared against the linear programming function to analyze if the active principle in within the standard boundaries or range for the medical product to be effective.

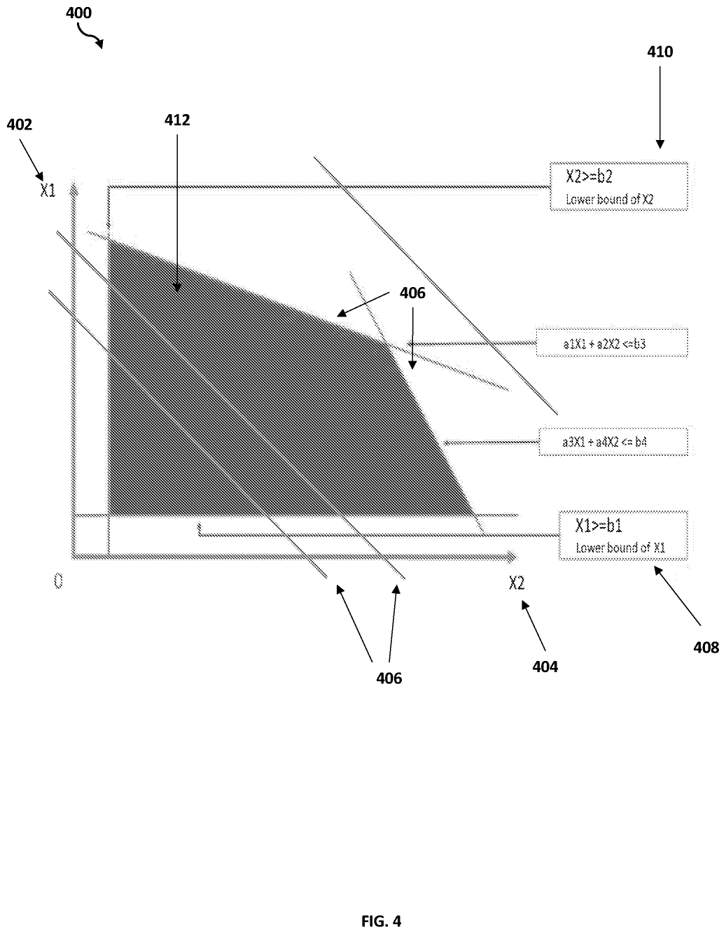

The results (e.g., the quantity of active principle in the current medical product compared to the standard medical product) may be presented to the user via the user device (e.g., dashboard on the user device). In at least one embodiment, the results may be presented as a numeric value in which the range of the standard medical product is indicated, and the quantity of active principle in the current medical product is presented. In at least one other embodiment, the results may be presented as a graphical representation. The exemplary graphical representation for a feasible solution from the optimization engine will be described in greater detail below with respect to FIG. 4.

In some embodiments, when the medical products includes multiple active principles, each of the active principles may be ranked or sorted based on the highest to lowest amount of each active principle may be included in a feasible solution of that medical product. As such, the results associated with active principle with the highest quantity may be presented first to the user, and so on until the active principle with the lowest quantity has been presented to the user. In at least one embodiment, the user, or an administrator may re-configure the consistency verification program 110a, 110b to modify how multiple active principles are ranked or sorted, as well as presented to the user.

Continuing with the previous example, the amounts in the two active principles are compared with the feasible solution for Medication XYZ, and the following is presented to User Z:

### Active Principle: Component Y

Feasible Solution: 149-250 mg

Current Medical Product: 150 mg

### Active Principle: Component Z

Feasible Solution: 400-500 mg

Current Medical Product: 140 mg

Then, at 212, the consistency verification program 110a, 110b determines whether the counterfeit risk is high. Based on whether the quantity of active principle of current medical product is within the range of the standard medical product (i.e., feasible solution), the consistency verification program 110a, 110b may simultaneously determine whether this is a high or low risk of counterfeit for the current medical product. As such, if the quantity of active principle is outside of the feasible solution (i.e., range of standard medical product), then there is a high counterfeit risk. However, if the quantity of active principle is inside the feasible solution, then there is a low counterfeit risk.

In another embodiment, the consistency verification program 110a, 110b may consecutively determine whether the counterfeit risk of the current medical product is high or low. As such, the consistency verification program 110a, 110b may present, to the user, the results of whether the current medical product falls within the feasible solution. After these results, the consistency verification program 110a, 110b may determine whether the counterfeit risk is high or low. The determination, by consistency verification program 110a, 110b, of the counterfeit risk may also be presented to the user thereafter. For example, the consistency verification program 110a, 110b determines that active principle Y₁ and Y₂ of Medical Product Y falls in the feasible solution, then the consistency verification program 110a, 110b will present such results, in numeric value, on the dashboard associated with the computing device of User B as follows:

### Active Principle: Y₁

Feasible Solution: 150-220 mg

Current Medical Product: 186 mg

### Active Principle: Y₂

Feasible Solution: 140-200 mg

Current Medical Product: 147 mg

Then, the consistency verification program 110a, 110b determines that the counterfeit risk is low, and then User B is presented with the following on the dashboard:

### Active Principle: Y₁

Counterfeit Risk: LOW

### Active Principle: Y₂

Counterfeit Risk: LOW

If the consistency verification program 110a, 110b determines that the counterfeit risk is high at 212, then the authorities are notified at 214. For any current medical product with a high counterfeit risk, the consistency verification program 110a, 110b may contact the appropriate authorities to report the current medical product. The consistency verification program 110a, 110b may provide the current medical product data to the appropriate authorities. In at least one embodiment, a list of appropriate authorities (e.g., local, regional, state, federal, international), which includes contact information (e.g., email address, name of contact representative for each authority, telephone number, mailing address, preferred method of contact, historical data associated with past notifications to such authority) may be previously compiled in the consistency verification program 110a, 110b, and stored in a contact information database (e.g., database 114).

In at least one embodiment, the historical data associated with past notifications to such authority may include the date of notification, date that the authority first reviewed the notification, date that the authority last reviewed or opened the notification, type of medical product that was subject that notification, current medical product data associated with the medical product that was the subject of the past notification (e.g., links to direct the user to the current medical product data), status of the notification (e.g., currently under investigation, closed, pending investigation, awaiting investigation, unviewed), and any resolution to this notification (e.g., date of resolution, details on the resolution). The consistency verification program 110a, 110b may utilize an external engine to search the historical data associated with past notifications to identify patterns or issues within the historical data. For example, if the authority has failed to review notifications for more than 30 days after date of notification, the user is informed of that delay. The user can determine whether the contact information should be modified for faster review of the notifications.

Based on the various factors associated with the current medical product data and the patient(s) (e.g., location of the administration, location of the patient, date of administration, age of the patient, type of medical product), the consistency verification program 110a, 110b may identify the appropriate authorities to be notified of such counterfeit risk. Then, the consistency verification program 110a, 110b may automatically notify the appropriate authorities based on the contact information provided in the previously compiled list of authorities. In at least one embodiment, the identified authority (or authorities) to be notified may be first presented to the user (e.g., via dialog box, or email). The user may first manually determine whether the identified authority should be notified due to the various factors associated with the current medical product data and the patient(s). The consistency verification program 110a, 110b may also present to the user the contact information for each approved authority before the authority is notified. The user may then edit, modify and/or approve the contact information to send the notification of such counterfeit risk on the current medical product.

Continuing with the previous example, since the feasible solution of Component Z is between 400-500 mg and Component Z in Medication XYZ is outside the feasible solution at 140 mg. Therefore, the consistency verification program 110a, 110b presents the following counterfeit risk for Component Z to User Z:

### Active Principle: Component Z

Counterfeit Risk: HIGH (outside Feasible Solution)

Therefore, the consistency verification program 110a, 110b identifies the three appropriate authorities due to the gender of Patient Z, the location of Company YZ and the location of Patient Z, and the consistency verification program 110a, 110b automatically notifies these authorities.

If, however, the consistency verification program 110a, 110b determines that the counterfeit risk is low at 212, then the consistency verification program 110a, 110b is concluded. Continuing with the previous example, since the feasible solution of Component Y is between 149-250 mg and Component Y in Medication XYZ is within the feasible solution at 150 mg. Therefore, the consistency verification program 110a, 110b presents the following counterfeit risk for Component Y to User Z:

### Active Principle: Component Y

Counterfeit Risk: LOW (within Feasible Solution)

Therefore, the consistency verification program 110a, 110b has concluded for Component Y since there is no reason to report a counterfeit risk for this Component Y to the authorities.

In another embodiment, regardless of whether the counterfeit risk is low or high, the consistency verification program 110a, 110b may identify any discrepancies in the current medical product data. The discrepancy may be mapped to create a hypothesis based on points of control and values, and may then generate alerts and guidance to notify the authorities, especially regulatory agencies. Continuing with the previous example, since the Company YZ produced far fewer active principles, Components Y and Z, than generally used to produce 15 million pills, there is a possibility that other Medication XYZ sold to other patients, may different quantity of Component Y. Therefore, the consistency verification program 110a, 110b may report the data collected for both active principles to the authorities.

In the present embodiment, the consistency verification program 110a, 110b may generate a report including the results for matching the overall quantity of the medical product produced with the active principles and/or key ingredients (or components) provided.

Referring now to FIG. 3, an operational flowchart illustrating the exemplary data engineering process 300 used by the consistency verification program 110a, 110b according to at least one embodiment is depicted.

At 302, the data wrangling stage is commenced. During the data wrangling stage, the consistency verification program 110a, 110b may utilize a crawl component to parse through different information source producers (e.g., Internet of Things (IoT), websites, images or tables from pamphlets, leaflets or brochures associated with the medical product). Based on key words (e.g., name of the medical product), the crawl component may utilize feature extraction techniques to identify features (e.g., data) associated with the medical product.

Once data associated with the medical product is identified, the crawl component may use a machine learning (ML) model to extract the context and information collected from the data by utilizing natural language processing (NLP) techniques for textual data and visual recognition techniques for image data. More specifically, for NLP, an external engine may utilize an NLP technique (e.g., structure extraction, language identification, tokenization, decompounding, lemmatization/stemming, acronym normalization and tagging, entity extraction, phrase extraction) to process the collected textual data. Then, individual words, phrases, and/or sentences, as well as the relationships between the individual words, phrases and/or sentences, may be extracted from the processed textual data by utilizing various extraction approaches (e.g., top down, bottoms up, statistical). As a result, the crawl component may interpret the context and meaning for the words, phrases and/or sentences collected by the textual data.

With image data, the crawl component may utilize one or more image recognition, visual recognition, and image processing tools (e.g., convolutional neural networks (CNNs), pattern recognition) to identify objects shown in an image. In various image recognition, visual recognition and image processing tools, an image may be broken down in a number of tiles that is individually analyzed, or classified into objects or classes based on key features, to determine the identity of the objects presented in the image.

In at least one embodiment, subject matter experts (SMEs) may be used to train the ML model that perform the NLP techniques, or visual recognition (including image recognition and image processing tools) for the textual and/or image data identified and extracted by the crawl component. The SMEs may extract the context to the unstructured information on the textual and/or image data associated with the medical product.

Then, during the data wrangling stage, the consistency verification program 110a, 110b may merge the unstructured information on the data collected (i.e., textual data and/or image data) associated with the medical product into one or more datasets.

Next, at 304, the data cleansing stage is commenced. During the data cleansing stage, the consistency verification program 110a, 110b may utilize an external engine (e.g., security information and event management (SIEM) or a customized data-science process that works through certain libraries associated with compatible programming languages) to parse the merged data sets and clean any missing, invalid, or inconsistent data values (i.e., erroneous data values) in the merged data sets. The cleansed data sets may then be syntactically correct and/or semantically correct, without outliers.

Then, at 306, the data preparation stage is commenced. During the data preparation stage, the consistency verification program 110a, 110b may utilize an external engine to normalize (or structure) the data (e.g., changing values into numerical values, where 0 represents false and 1 represents true). Therefore, the external engine may transform the data on each dimension for each data domain into a valid input for a ML model supervised during the training phase of the consistency verification program 110a, 110b at 208.

FIG. 4 is an exemplary graphical representation 400 illustrating for a feasible solution for multiple active principles in a medical product from an optimization engine during the optimization phase by the consistency verification program 110a, 110b according to at least one embodiment is depicted.

As shown in FIG. 4, the active principles, x₁ and x₂ within Medical Product X, are defined by a line and the feasible solution is defined based on the constraints equations. At 402, active principle x₁ is on the y-axis in which the amount of x₁ increases as the amount of x₁ moves away from 0, and at 404, active principle x₂ is on the x-axis in which the x₂ increases similarly as the amount of x₂ moves away from 0. Lines 406 represents each constraint on the active principles in which 408 is the lower bounds (or quantity) for active principle x₁ and 410 is the lower bounds (or quantity) for active principle x₂. The feasible solution of Medical Product X with the active principles x₁ and x₂ are indicated by 412 based on the various constraints of the active principles to make an effective Medical Product X. As such, any amount of active principles x₁ and x₂ that falls outside of the feasible solution is any combination of active principles x₁ and x₂ that falls outside of 412.

As such, if a solution (i.e., current medical product) is outside the feasible solution of 412 considering the constraints delivered by the trained ML models, the consistency verification program 110a, 110b may determine that there is a high counterfeit risk associated with Medical Product X. This graphical representation may be displayed in a dashboard to the user of the consistency verification program 110a, 110b. The graphical representation may further be a source of information for an investigation on Medical Product X.

The consistency verification program 110a, 110b may improve the functionality of the computer, the technology and/or the field of technology by determining a risk of a medical product being counterfeit based on the amount of active principle (or active pharmaceutical ingredient) from a predetermined quantity of the medical product produced based on the aggregated data from various sources utilizing a trained supervised machine learning (ML) model. The consistency verification program 110a, 110b may verify the active principles (or key components) of a medical product by evaluating component details, and checking the industry capacity to produce those key components (i.e., active principle) to confirm and assure that the industry may be capable to produce the volume according to the component delivered.

The consistency verification program 110a, 110b further may analyze such information (or data) collected by Internet of Things (IoT), websites and other sources, extracting the context/meaning of the information collected using feature extraction techniques, NLP techniques and visual recognition techniques for non-structured data, treating the data in such a way that may facilitate the data processing. Then, using the supervised ML model, the consistency verification program 110a, 110b may separate each dimension, where each one may be trained separately to improve the results and the optimization engine may be utilized to reduce failures and improve precision, as well as to identify where the counterfeit risk is high or low.

Additionally, the consistency verification program 110a, 110b may evaluate (or validate) the possibility of a counterfeit medical product based on the quantity and/or quality of the active principle included in a current medical product. The consistency verification program 110a, 110b may identify discrepancies in the quality and quantity of the active principles of a current medical product based on the data from different sources, and may determine whether there is a risk of counterfeit. The consistency verification program 110a, 110b may then notify the appropriate authorities, if applicable.

It may be appreciated that FIGS. 2-4 provide only an illustration of one embodiment and do not imply any limitations with regard to how different embodiments may be implemented. Many modifications to the depicted embodiment(s) may be made based on design and implementation requirements.

FIG. 5 is a block diagram 900 of internal and external components of computers depicted in FIG. 1 in accordance with an illustrative embodiment of the present invention. It should be appreciated that FIG. 5 provides only an illustration of one implementation and does not imply any limitations with regard to the environments in which different embodiments may be implemented. Many modifications to the depicted environments may be made based on design and implementation requirements.

Data processing system 902, 904 is representative of any electronic device capable of executing machine-readable program instructions. Data processing system 902, 904 may be representative of a smart phone, a computer system, PDA, or other electronic devices. Examples of computing systems, environments, and/or configurations that may represented by data processing system 902, 904 include, but are not limited to, personal computer systems, server computer systems, thin clients, thick clients, hand-held or laptop devices, multiprocessor systems, microprocessor-based systems, network PCs, minicomputer systems, and distributed cloud computing environments that include any of the above systems or devices.

User client computer 102 and network server 112 may include respective sets of internal components 902a, b and external components 904a, b illustrated in FIG. 5. Each of the sets of internal components 902a, b includes one or more processors 906, one or more computer-readable RAMs 908 and one or more computer-readable ROMs 910 on one or more buses 912, and one or more operating systems 914 and one or more computer-readable tangible storage devices 916. The one or more operating systems 914, the software program 108, and the consistency verification program 110a in client computer 102, and the consistency verification program 110b in network server 112, may be stored on one or more computer-readable tangible storage devices 916 for execution by one or more processors 906 via one or more RAMs 908 (which typically include cache memory). In the embodiment illustrated in FIG. 5, each of the computer-readable tangible storage devices 916 is a magnetic disk storage device of an internal hard drive. Alternatively, each of the computer-readable tangible storage devices 916 is a semiconductor storage device such as ROM 910, EPROM, flash memory or any other computer-readable tangible storage device that can store a computer program and digital information.

Each set of internal components 902a, b also includes a R/W drive or interface 918 to read from and write to one or more portable computer-readable tangible storage devices 920 such as a CD-ROM, DVD, memory stick, magnetic tape, magnetic disk, optical disk or semiconductor storage device. A software program, such as the software program 108 and the consistency verification program 110a, 110b can be stored on one or more of the respective portable computer-readable tangible storage devices 920, read via the respective R/W drive or interface 918 and loaded into the respective hard drive 916.

Each set of internal components 902a, b may also include network adapters (or switch port cards) or interfaces 922 such as a TCP/IP adapter cards, wireless wi-fi interface cards, or 3G or 4G wireless interface cards or other wired or wireless communication links. The software program 108 and the consistency verification program 110a in client computer 102 and the consistency verification program 110b in network server computer 112 can be downloaded from an external computer (e.g., server) via a network (for example, the Internet, a local area network or other, wide area network) and respective network adapters or interfaces 922. From the network adapters (or switch port adaptors) or interfaces 922, the software program 108 and the consistency verification program 110a in client computer 102 and the consistency verification program 110b in network server computer 112 are loaded into the respective hard drive 916. The network may comprise copper wires, optical fibers, wireless transmission, routers, firewalls, switches, gateway computers and/or edge servers.

Each of the sets of external components 904a, b can include a computer display monitor 924, a keyboard 926, and a computer mouse 928. External components 904a, b can also include touch screens, virtual keyboards, touch pads, pointing devices, and other human interface devices. Each of the sets of internal components 902a, b also includes device drivers 930 to interface to computer display monitor 924, keyboard 926 and computer mouse 928. The device drivers 930, R/W drive or interface 918 and network adapter or interface 922 comprise hardware and software (stored in storage device 916 and/or ROM 910).

It is understood in advance that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g. networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Analytics as a Service (AaaS): the capability provided to the consumer is to use web-based or cloud-based networks (i.e., infrastructure) to access an analytics platform. Analytics platforms may include access to analytics software resources or may include access to relevant databases, corpora, servers, operating systems or storage. The consumer does not manage or control the underlying web-based or cloud-based infrastructure including databases, corpora, servers, operating systems or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure comprising a network of interconnected nodes.

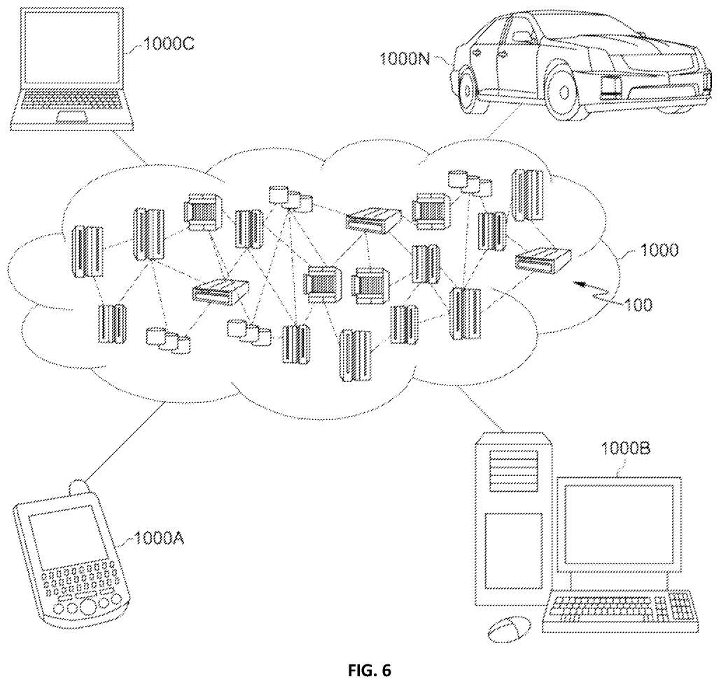

Referring now to FIG. 6, illustrative cloud computing environment 1000 is depicted. As shown, cloud computing environment 1000 comprises one or more cloud computing nodes 100 with which local computing devices used by cloud consumers, such as, for example, personal digital assistant (PDA) or cellular telephone 1000A, desktop computer 1000B, laptop computer 1000C, and/or automobile computer system 1000N may communicate. Nodes 100 may communicate with one another. They may be grouped (not shown) physically or virtually, in one or more networks, such as Private, Community, Public, or Hybrid clouds as described hereinabove, or a combination thereof. This allows cloud computing environment 1000 to offer infrastructure, platforms and/or software as services for which a cloud consumer does not need to maintain resources on a local computing device. It is understood that the types of computing devices 1000A-N shown in FIG. 6 are intended to be illustrative only and that computing nodes 100 and cloud computing environment 1000 can communicate with any type of computerized device over any type of network and/or network addressable connection (e.g., using a web browser).

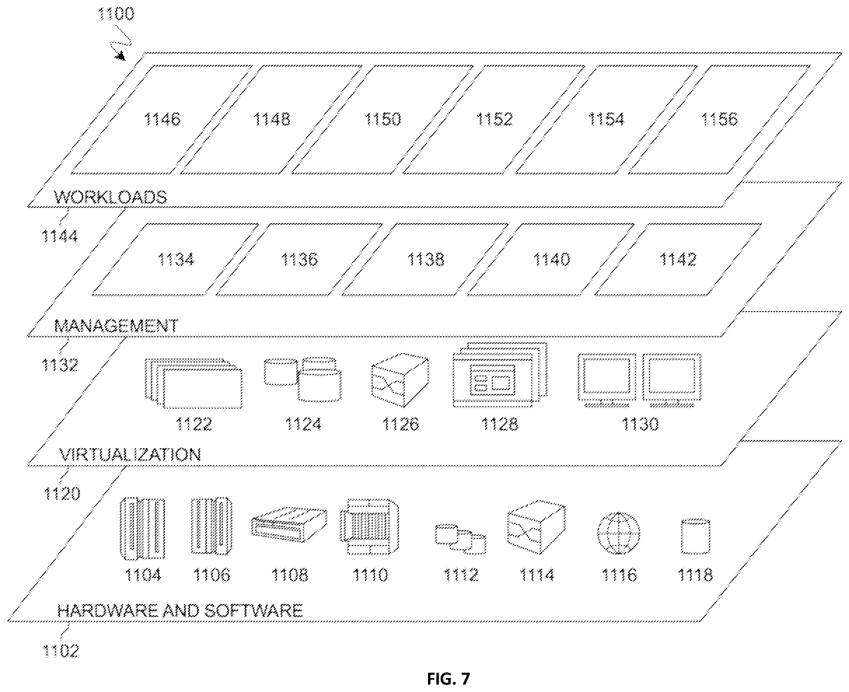

Referring now to FIG. 7, a set of functional abstraction layers 1100 provided by cloud computing environment 1000 is shown. It should be understood in advance that the components, layers, and functions shown in FIG. 7 are intended to be illustrative only and embodiments of the invention are not limited thereto. As depicted, the following layers and corresponding functions are provided:

Hardware and software layer 1102 includes hardware and software components. Examples of hardware components include: mainframes 1104; RISC (Reduced Instruction Set Computer) architecture based servers 1106; servers 1108; blade servers 1110; storage devices 1112; and networks and networking components 1114. In some embodiments, software components include network application server software 1116 and database software 1118.

Virtualization layer 1120 provides an abstraction layer from which the following examples of virtual entities may be provided: virtual servers 1122; virtual storage 1124; virtual networks 1126, including virtual private networks; virtual applications and operating systems 1128; and virtual clients 1130.

In one example, management layer 1132 may provide the functions described below. Resource provisioning 1134 provides dynamic procurement of computing resources and other resources that are utilized to perform tasks within the cloud computing environment. Metering and Pricing 1136 provide cost tracking as resources are utilized within the cloud computing environment, and billing or invoicing for consumption of these resources. In one example, these resources may comprise application software licenses. Security provides identity verification for cloud consumers and tasks, as well as protection for data and other resources. User portal 1138 provides access to the cloud computing environment for consumers and system administrators. Service level management 1140 provides cloud computing resource allocation and management such that required service levels are met. Service Level Agreement (SLA) planning and fulfillment 1142 provide pre-arrangement for, and procurement of, cloud computing resources for which a future requirement is anticipated in accordance with an SLA.

Workloads layer 1144 provides examples of functionality for which the cloud computing environment may be utilized. Examples of workloads and functions which may be provided from this layer include: mapping and navigation 1146; software development and lifecycle management 1148; virtual classroom education delivery 1150; data analytics processing 1152; transaction processing 1154; and consistency verification 1156. A consistency verification program 110a, 110b provides a way to verify the consistency of a medical product based on the quantity and/or quality of the active principle.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A computer-implemented method comprising:
generating a quantity associated with each of one or more active principles in a current medical product based on a plurality of current medical product data and a plurality of information from a plurality of data domains;
comparing the generated quantity associated with each of the one or more active principles in the current medical product with one or more constraints associated with a feasible solution associated with the current medical product; and
determining a level of counterfeit risk based on the compared quantity associated with each of the one or more active principles in the current medical product with the one or more constraints from the feasible solution associated with the current medical product.

2. The method of claim 1, further comprising:
presenting, to a user, a plurality of results associated with the compared quantity associated one or more active principles in the current medical product with the one or more constraints from the feasible solution for the current medical product.

3. The method of claim 2, further comprising:
identifying one or more discrepancies associated with the one or more active principles from the current medical product; and
notifying one or more authorities on the identified one or more discrepancies.

4. The method of claim 2, further comprising:
in response to determining that the compared quantity associated with the one or more active principles in the current medical product are outside of the one or more constraints from the feasible solution for the current medical product, determining the level of counterfeit risk is high; and
notifying one or more authorities that the compared quantity associated with the one or more active principles in the current medical product are outside of the feasible solution for the current medical product.

5. The method of claim 2, further comprising:
in response to determining that the compared quantity associated one or more active principles in the current medical product are inside of the one or more constraints from the feasible solution associated for the current medical product, determining the level of counterfeit risk is low.

6. The method of claim 1, further comprising:
parsing through a set of unstructured data from a plurality of information source producers;
identifying one or more features associated with the current medical product from the parsed unstructured data by utilizing one or more feature extraction techniques;
extracting a plurality of context associated with the identified one or more features by utilizing natural language processing (NLP) techniques and visual recognition techniques;
merging the set of unstructured data associated with the extracted plurality of context from the identified one or more features into one or more datasets;
cleansing the one or more datasets,
wherein one or more sets of erroneous data values are eliminating;
generating the cleansed one or more datasets in absence of one or more outliers,
wherein the generated one or more datasets are syntactically correct and semantically correct; and
normalizing the generated one or more datasets.

7. The method of claim 6, further comprising:
training a machine learning (ML) model for each dimension associated with each data domain,
wherein a logistic regression algorithm is utilized,
wherein one or more output values associated with each of the trained ML model is a coefficient for each dimension,
wherein each coefficient includes an acceptable limit for the one or more active principles associated with the current medical product; and
building an objective function from each coefficient for each of the one or more active principles in the current medical product.

8. A computer system for verifying a consistency of a current medical product, comprising:
one or more processors, one or more computer-readable memories, one or more computer-readable tangible storage medium, and program instructions stored on at least one of the one or more tangible storage medium for execution by at least one of the one or more processors via at least one of the one or more memories, wherein the computer system is capable of performing a method comprising:
generating a quantity associated with each of one or more active principles in the current medical product based on a plurality of current medical product data and a plurality of information from a plurality of data domains;
comparing the generated quantity associated with each of the one or more active principles in the current medical product with one or more constraints associated with a feasible solution associated with the current medical product; and
determining a level of counterfeit risk based on the compared quantity associated with each of the one or more active principles in the current medical product with the one or more constraints from the feasible solution associated with the current medical product.

9. The computer system of claim 8, further comprising:
presenting, to a user, a plurality of results associated with the compared quantity associated one or more active principles in the current medical product with the one or more constraints from the feasible solution for the current medical product.

10. The computer system of claim 9, further comprising:
identifying one or more discrepancies associated with the one or more active principles from the current medical product; and
notifying one or more authorities on the identified one or more discrepancies.

11. The computer system of claim 9, further comprising:
in response to determining that the compared quantity associated with the one or more active principles in the current medical product are outside of the one or more constraints from the feasible solution for the current medical product, determining the level of counterfeit risk is high; and
notifying one or more authorities that the compared quantity associated with the one or more active principles in the current medical product are outside of the feasible solution for the current medical product.

12. The computer system of claim 9, further comprising:
in response to determining that the compared quantity associated one or more active principles in the current medical product are inside of the one or more constraints from the feasible solution associated for the current medical product, determining the level of counterfeit risk is low.

13. The computer system of claim 8, further comprising:
parsing through a set of unstructured data from a plurality of information source producers;
identifying one or more features associated with the current medical product from the parsed unstructured data by utilizing one or more feature extraction techniques;
extracting a plurality of context associated with the identified one or more features by utilizing natural language processing (NLP) techniques and visual recognition techniques;
merging the set of unstructured data associated with the extracted plurality of context from the identified one or more features into one or more datasets;
cleansing the one or more datasets,
wherein one or more sets of erroneous data values are eliminating;
generating the cleansed one or more datasets in absence of one or more outliers,
wherein the generated one or more datasets are syntactically correct and semantically correct; and
normalizing the generated one or more datasets.

14. The computer system of claim 13, further comprising:
training a machine learning (ML) model for each dimension associated with each data domain,
wherein a logistic regression algorithm is utilized,
wherein one or more output values associated with each of the trained ML model is a coefficient for each dimension,
wherein each coefficient includes an acceptable limit for the one or more active principles associated with the current medical product; and
building an objective function from each coefficient for each of the one or more active principles in the current medical product.

15. A computer program product for verifying a consistency of a current medical product, comprising:
one or more computer-readable storage media and program instructions stored on at least one of the one or more tangible storage media, the program instructions executable by a processor to cause the processor to perform a method comprising:
generating a quantity associated with each of one or more active principles in the current medical product based on a plurality of current medical product data and a plurality of information from a plurality of data domains;
comparing the generated quantity associated with each of the one or more active principles in the current medical product with one or more constraints associated with a feasible solution associated with the current medical product; and
determining a level of counterfeit risk based on the compared quantity associated with each of the one or more active principles in the current medical product with the one or more constraints from the feasible solution associated with the current medical product.

16. The computer program product of claim 15, further comprising:
presenting, to a user, a plurality of results associated with the compared quantity associated one or more active principles in the current medical product with the one or more constraints from the feasible solution for the current medical product.

17. The computer program product of claim 16, further comprising:
identifying one or more discrepancies associated with the one or more active principles from the current medical product; and
notifying one or more authorities on the identified one or more discrepancies.

18. The computer program product of claim 16, further comprising:
in response to determining that the compared quantity associated with the one or more active principles in the current medical product are outside of the one or more constraints from the feasible solution for the current medical product, determining the level of counterfeit risk is high; and
notifying one or more authorities that the compared quantity associated with the one or more active principles in the current medical product are outside of the feasible solution for the current medical product.

19. The computer program product of claim 16, further comprising:
in response to determining that the compared quantity associated one or more active principles in the current medical product are inside of the one or more constraints from the feasible solution associated for the current medical product, determining the level of counterfeit risk is low.

20. The computer program product of claim 15, further comprising:
parsing through a set of unstructured data from a plurality of information source producers;
identifying one or more features associated with the current medical product from the parsed unstructured data by utilizing one or more feature extraction techniques;
extracting a plurality of context associated with the identified one or more features by utilizing natural language processing (NLP) techniques and visual recognition techniques;
merging the set of unstructured data associated with the extracted plurality of context from the identified one or more features into one or more datasets;
cleansing the one or more datasets,
wherein one or more sets of erroneous data values are eliminating;
generating the cleansed one or more datasets in absence of one or more outliers,
wherein the generated one or more datasets are syntactically correct and semantically correct; and
normalizing the generated one or more datasets.
